(19) United States
(12) Patent Application Publication
(10) Pub. No.: US 2004/0058856 A1
(43) Pub. Date: Mar. 25, 2004

(54) Oligolysine transducing domain, oligolysine-cargo molecule complex and uses thereof

(76) Inventors: Soo-Young Choi, Chuncheon-shi (KR); Jinseu Park, Chuncheon-shi (KR); Hyeok-Yil Kwon, Chuncheon-shi (KR); Jung-Hoon Kang, Cheongju-shi (KR); Tae-Chun Kang, Chuncheon (KR); Moo-Ho Won, Chuncheon (KR); Kyu-Hyung Han, Chuncheon-shi (KR); Kil-Soo Lee, Seoul (KR)

Correspondence Address: FITZPATRICK CELLA HARPER & SCINTO, 30 ROCKEFELLER PLAZA, NEW YORK, NY, 10112, US

(21) Appl. No.: 10/333,578

(22) PCT Filed: May 21, 2001
(86) PCT No.: PCT/KR01/00835

(30) Foreign Application Priority Data
Jul. 26, 2000 (KR) 2000/043022
Feb. 8, 2001 (KR) 2001/006178
Mar. 3, 2001 (KR) 2001/010981
Mar. 19, 2001 (KR) 2001/014147

(51) Int. Cl. A61K 48/00; A61K 38/16; C07K 14/00; C12N 15/85

(52) U.S. Cl. 514/7; 530/395; 435/455

(57) ABSTRACT

This invention relates to delivery of biologically active organic molecules e.g. peptides or proteins into the cytoplasm. In this invention, transducing domain which is covalently attached to organic molecules is oligolysine which comprise 3-12 lysine residues. The oligolysine transducing domain-binding fusion protein is efficiently transducible into cytoplasm, and biologically active.

### TECHNICAL FIELD

The present invention relates to oligolysine transport domain, an oligolysine vector, oligolysine-cargo molecule complex, each of which being comprised of a plurality of lysine residues, an expression vector of the complex and a method for transducing the complex into cells and an use thereof.

### BACKGROUND ART

As it is at present known that various diseases people gets are due to the abnormal activity of cell protein, many studies for developing medicines capable of treating serious diseases by adjusting the activity of the protein have been made all over the world. Thereby, development for regulatory materials of a peptide or protein type has been rapidly endeavored on the basis of enzyme and protein that can regulate the particular physiological activity within the cell and the cooperative structure between the protein. However, even though the peptide, polypeptide and protein exhibit more excellent selectivity and efficiency in the particular physiological activity than other compounds, they are substantially difficult to pass through the cell membrane because of their size or biochemical properties they have so that they fail to be carried in the interior of the cell. As a result, they cannot be functioned as an effective treatment agent or a basic research means. Generally, it is noted that a material having molecular weight of 600 or more does not almost pass through the cell membrane (For example, see Schwartze et al., 1999; Van de Waterbeemd et al., 1998).

Various methods for carrying the protein, polypeptide and peptide to the cell have been tried. Examples of the methods are physical treatment (e.g., microinjection, scrape loading, electroperforation and biolistics), formation of pores on the cell in a chemical or biological manner (e.g., digitonin, pore-forming protein and ATP treatment), employment of modified protein or protein carriers (e.g., lipidated protein, immunotoxins and related bioconjugates), and particles engulfment or fusion (e.g., liposomes, cell-cell fusion, virus mimics and induced pinocytosis) (For example, see Fernandez and Bayley, 1998). However, such the methods as discussed above have the following problems: For example, the microinjection treatment is advantageously carried out only when a single cell or a small number of cells are studied; and the immunotoxins treatment fails not only to carry normal protein to the cell at a high degree but also to make a target cell killed at the time of using the protein having fatal activity. In addition, the above-discussed methods have experienced some problems such as, for example, the failure of the carrying of protein to cell, the damage of the cell, the non-arrival of target protein, the inconsitent results.

Recently, it is found that Tat (Transactivator of transcription) protein as one of protein expressed by Human Immunodeficiency Virus type-1 (HIV-1) passes effectively through a cell membrane and easily moves into the cytoplasm and cell nucleus (For example, see Frankel and Pabo, 1998; Green and Loewenstein, 1988). Also, it is found that when a different kind of protein such as ovalbumin, β-galactosidase, horseradish peroxidase is fused with the HIV-1 Tat protein and applied, it is directly carried to each tissue of a living body and cultivated cells (For example, see Fawell et al., 1994; Schwartze et al., 1999). It is known that such the function is implemented by a basic domain, that is, a protein transduction domain positioned at the intermediate portion of the Tat protein (For example, see Nagahara et al., 1998; Vives et al., 1997). Besides the HIV-1 Tat protein, it is reported that peptides originated from an anntennapedia homeodomain can carry protein to a cell (For example, see Derossi et al., 1996). The mechanism of the protein transduction to the cell is not exactly suggested in the above-discussed document, but it can be understood that the transducing domain of the protein acts directly on the lipid layer of the cell membrane, without having specific receivers or carriers.

The peptides having such the capability of carrying the protein to the cell include 10 to 16 amino acid residues, and it is known they are containing a lot of the arginine or lysine residues each having a positive electric charge are plenty. Therefore, the present inventors have checked that several to tens of lysine residues are covalently bonded with the protein such that the protein is carried to the cell.

Typically, a polylysine made by the polymerization of tens or hundreds of lysine residues is used as a carrying material for carrying nucleic acid to a cell. However, the conventional polylysine comprised of the tens of lysine residues or more does not exist at the covalent bonded state with the nucleic acid but is bonded with the nucleic acid by coating a negative electric charge prohibiting the nucleic acid from passing through the cell membrane.

Therefore, the present inventors have made various studies to develop the voluntary carrying of protein to a cell and as a result, they have found that a vector that expresses oligolysine fusion protein to which several to tens of lysine residues are attached is produced and the expressed oligolysine fusion protein is carried effectively to the cell and keeps its activity in the cell.

### DISCLOSURE OF INVENTION

Accordingly, the present invention is directed to an oligolysine carrying domain that substantially obviates one or more problems due to limitations and disadvantages of the related art.

An object of the present invention is to provide an oligolysine carrying domain that is comprised of a plurality of lysine residues capable of carrying protein to a cell and thus covalently bonded with biologically active protein to be thereby expressed as oligolysine fusion protein to be carried to the cell.

Another object of the present invention is to provide pharmaceutical composition and cosmetic compositions having oligolysine fusion protein as an effective component.

### BRIEF DESCRIPTION OF THE DRAWINGS

The above objectives and advantages of the present invention will become more apparent by describing in detail of preferred embodiments thereof with reference to the attached drawings in which:

FIG. 1 is showing an expression vector of fusion protein on which is attached oligolysine. This vector could express 6 histidines, 9 lysine residues (Hereinafter will be referred to as “9K”, “K9” or “Lys.”) and target protein into fusion protein. That is, the fusion protein will be expressed in order of the 6 histidines, 9 lysine residues and target protein; starting from amino terminus,

FIG. 2 is a graph showing analyzed results of protein produced from fusion protein expression vector of FIG. 1. The fusion protein is expressed from Escherichia coli (E. coli), and purified by a PD-10 chromatography comprising Ni-NTA column and Sephadex G-25M. (A) is a typical diagram showing a process of purifying Lys-GFP fusion protein in partial denaturation condition, and (B) is a graph showing analyzed results of Lys-GFP fusion protein and control GFP by SDS-PAGE. (C) is a graph showing analyzed results of Lys-GFP fusion protein and control GFP by western blotting;

FIG. 3 is showing a comparison result of cell transducing efficiency between Lys-GFP fusion protein purified in denaturing condition and native condition respectively. Each protein is treated in HeLa cell for 2 hrs in 0.5 μM concentration:

Lane 1: Control GFP;

Lane 2: Lys-GFP fusion protein in native condition; and

Lane 3: Lys-GFP fusion protein in denaturation condition;

FIG. 4 is a graph showing comparison results of transducing efficiency from fusion proteins to eucaryotic cell by a protein concentration, treatment time:

(A) Purified Lys-GFP treated for 2 hrs in a HeLa cell by concentration and then analyzed with Western blotting;

(B) Lys-GFP fusion protein of 0.5 μM concentration treated in HeLa cell by time;

FIG. 5 is a graph showing an activity of fusion protein transduced into a cell, in which a Lys-GFP fusion protein and control GFP is treated in HeLa cell and analyzed of their fluorescence rate with fluorescence microscope;

FIG. 6 illustrates a sequence of polynucleotide coding a Lys-GFP protein;

FIG. 7 is showing the purified Lys-GFP which is analyzed by SDS-PAGE;

FIG. 8 is a graph showing fluorescent analysis of the transducing efficiency according to a concentration of Lys-GFP fusion protein;

FIG. 9 is showing an analysis result of Lys-GFP fusion protein analyzed by Western blotting;

FIG. 10 is showing a result of rat skin cell penetration experiment. A and B are indicating epidermis and dermis of control group and experimental group respectively, while C and D are indicating subcutaneous adipose tissues of the control group and experimental group respectively. An arrow in the figure is indicating color reaction site;

FIG. 11 is showing a penetration efficiency, analyzed West blotting, of Lys-SOD and HIV-1 Tat (49-57)-SOD into HeLa cell, by concentration;

FIG. 12 is a diagram illustrating a preparing process of Lys-CAT expression vector. A shows the preparing process of pLys-CAT expression vector by using pET15b vector, and B shows a diagram of the expressed fusion protein Lys-CAT and CAT (catalase) used as a control protein;

FIG. 13 is showing an activity of protein with specific activity graph by analyzing Lys-catalase penetrated into PC12 cell according to the change of concentration by Western blotting. A is showing the result of analyzing a fusion protein penetrated into the cell after 3 hrs elapsed from an administration of 0.5˜4 μM Lys-catalase and catalase into cell culture solution. B is showing activity of recombinant protein penetrated into the cell;

FIG. 14 is showing Lys-CAT penetrated into PC12 cell by time and an activity thereof. A is showing the measuring result of fusion protein, which is penetrated into the cell after treating 4 μM of recombinant protein for 0.5 to 4 hrs, by Western blotting and activity (14B) analysis;

FIG. 15 is showing a stability of Lys-CAT penetrated into PC12 cell. To measure the stability of Lys-CAT penetrated into PC12 cell, 4 μM of Lys-CAT is administered into a cell culture solution and the amount of fusion protein which remained in the cell is analyzed by Western blotting (15A) and activity analysis (15B) by time (0˜72 hrs);

FIG. 16 is a graph showing the effects of Lys-CAT penetrated into the cell on the cell survival. To measure the effects of Lys-CAT penetrated into the cell on the cell survival, when hydrogen peroxide is added from the exterior, Lys-CAT (0.1-4 uM) and CAT are administered into a cell culture solution and hydrogen peroxide was added at 6 hrs later, and MTT assay was conducted;

FIGS. 17 and 18 are showing skin penetration efficiency of Lys-CAT. 0.3 mg of CAT and Lys-CAT each are coated on the skin of abdominal region and slaughtered by time (30 mins, 1 hrs, 2 hrs) to measure the activity of protein penetrated into tissue (FIG. 18), which then is analyzed of the penetration efficiency into the skin by immunohistochemical dye staining test (FIG. 17); and

FIG. 19 is a sequence of recombinant polynucleotide coding Lys-CAT.

### BEST MODE FOR CARRYING OUT THE INVENTION

The present invention relates to a method for efficiently delivering biologically active proteins, nucleic acids and other molecules not easily capable of penetrating or naturally entering at an effective rate into a target cell or cell nucleus. The delivering of cargo molecule into the cell according to the present invention is performed through oligolysine transport domain consisting of several to dozens of lysine residues. Specifically, the present invention relates to a novel oligolysine transport domain, a method for preparing the domain, an oligolysine transport domain-cargo molecule complex, and a pharmaceutical composition for treating, preventing and diagnosing and cosmetic composition comprising the oligolysine transport domain-cargo molecule complex.

The definitions of the terms as used herein are as follows.

The term “cargo molecule” refers to, a molecule per se prior to being fused to the transport domain as a molecule, which is naturally not capable of penetrating at an effective rate into the target cell, but not a transport domain or a part thereof, or cargo molecule part of the transport domain-cargo molecule complex. As cargo molecules, polypeptides, proteins, nucleic acids, polysaccharides, and other organic compounds are included. The “cargo molecule” also refers to, for example, drug, receptor, etc, in the usage or application of the present invention. The “cargo molecule” herein has the same or analogous meaning to “target protein”, etc.

The term “oligolysine (transport domain)-cargo molecule complex” means a complex formed by genetic fusion or chemical bond between the transport domain and cargo molecule, and includes the transport domain and at least one cargo molecule part.

Also, said “genetic fusion” means a linkage by the linear, covalent bond formed through the genetic expression of DNA sequence encoding a protein.

Further, the term “target cell” means a cell into which the cargo molecule is delivered by the transport domain, and refers to an in vivo or ex vivo cell. That is, the target cell has the meaning including in vivo cell, so to speak, a cell that constitutes organs or tissues of living animal or human, or a microorganism found in living animal or human. The target cell also includes ex vivo cell, that is, cultured animal cell, human cell or microorganism.

The “oligolysine transport domain” herein consists of several to dozens of lysine residues which can covalently be bound with high molecular organic compounds, e.g., oligonucleotides, peptides, proteins, oligosaccharides, polysaccharides, etc, thereby introducing said organic compounds into the cell having no need of any receptors, carriers or energy.

The term “cargo protein” or “target protein” used herein refers to a treatment molecule, prevention molecule, diagnosis molecule and the like which can form a covalent bond with said oligolysine transport domain, thereby being introduced into the cell and exhibits activities. Indeed, they are not limited to a pure protein, but include peptides, polypeptides, glycoproteins bounded with saccharides, peptidoglycan and the like.

In addition, the expression “introducing” of proteins, peptides, organic compounds, etc, into the cell are used together with “carrying”, “penetrating”, “transducing”, “transporting”, “delivering”, “permeating” and “transiting”.

The present invention provides an oligolysine (used together with “9K” and “Lys” in the specification and drawings) transport domain which can carry the cargo molecule comprising various high-molecular and low-molecular organic compounds, which function in a cell, to the target cell, and which consists of various lysine residues in order to show activities in the cell; a vector comprising the oligolysine transport domain; an oligolysine-cargo molecule complex comprising oligolysine fusion protein produced by using the vector; a method for introducing the complex into the cell; and use thereof.

The oligolysine-cargo molecule complex and the like which introduce the selected specific “protein” as a cargo molecule into the cell are explained in the following detailed description. However, These are provided solely for convenience and should not be restricted the cargo molecule to protein or be interpreted to limit the scope of the invention to the oligolysine fusion protein and the like.

In order to develop a method for penetrating molecules such as protein and peptide having a specific function into the cell, the present inventors prepared a pLys as a transport domain-cargo molecule complex expression vector that can deliver the cargo molecule and/or target protein to the target cell. The newly produced vector was designed so as to express in the form of fusion protein comprising six histidines, 3 to 12 lysine residues and the target protein, in the order of starting from amino terminus, as the cargo molecule. To easily analyze a capability of oligolysine part for delivering the cargo molecule such as protein into the cell, after selecting GFP (Green Fluorescence Protein) as the target protein and inserting the DNA fragment corresponding to base sequence of GFP into pLys vector, pLys-GFP vector which can express Lys-GFP fusion protein and Lys-GFP fusion protein were produced.

For the purpose of comparison with intracellular permeability, oligolysine-GFP fusion protein was purified under the denaturing condition and native condition, respectively. A cell was subject to ultra-sonication in solution containing 6M urea, purified under by Ni-nitrilotriacetic acid sepharose column using six histidine residues, and salts were removed therefrom to obtain a partially denatured fusion protein.

In order to investigate an intracellular delivery effect of the oligolysine-GFP fusion protein thus purified under the denaturing condition and native condition, respectively, each oligolysine-GFP fusion protein purified under the denaturing condition and native condition was treated in cell culture solution at a concentration of 0.5 μM for 2 hours. As a result, it was observed by Western blotting that the oligolysine-GFP fusion protein purified under the denaturing condition was delivered into the cell with greater efficiency, compared to the oligolysine-GFP fusion protein purified under the native condition, whereas the control GFP used as a control protein did not penetrate into cell membrane at all.

Also, the present inventors analyzed the ratio of delivered cell when fusion protein was delivered into the target cell and the maintenance degree of activity peculiar to the fusion protein delivered into the cell. The degree of fluorescence of GFP in denatured oligolysine-GFP fusion protein-treated HeLa cell was observed by fluorescence microscope. The denatured oligolysine-GFP fusion protein was delivered into HeLa cell at the rate of almost 100%, and the recovery of GFP protein activity in the cytoplasm after the penetration into HeLa cell was observed.

The present inventors produced an Lys-CAT (hereinafter, refers to fusion protein catalase which forms a covalent bond with oligolysine consisting of nine lysine residues, and used together with “Lys-Catalase”, “oligolysine-Catalase”, “Lys-Catalase”) bounded with the oligolysine transport domain and Lys-SOD fusion protein by selecting a catalase (hereinafter, used together with “CAT”) and Cu, Zn-superoxide dismutase (hereinafter, used together with “SOD”) showing removal capability against reactive oxygen species as cargo molecules, and measured the penetration capability into cell and activity in cell with regard to the fusion protein thus produced.

The pharmaceutical composition containing oligolysine bound fusion protein as an effective ingredient can be formulated in the form for oral or injection together with pharmaceutically acceptable carriers in a conventional manner. As compositions for oral administration, for example, tablets and gelatin capsules are exemplified, and may further contain additives selected from the group consisting of diluents (e.g., lactose, dextrose, sucrose, mannitol, sorbitol, cellulose and/or glycine), lubricants (e.g., silica, talc, stearic acid and magnesium or calcium salt thereof and/or polyethylene glycol) in addition to the effective ingredient. In case of tablets, the composition contains binders (e.g., magnesium aluminum silicate, starch paste, gelatin, methyl cellulose, sodium carboxymethylcellulose and/or polyvinylpyrrolidone), and preferably disintegrates (e.g., starch, agar, alginic acid or sodium salt thereof) and/or absorbents, colorants, flavors and sweeteners. The composition for injection is preferably made in the form of isotonic solution or suspension, and the composition is sterilized and/or contains proper adjuvants (e.g., preservatives, stabilizers, wetting agents or dispersion promoters, salts for the regulation of osmotic pressure or buffers). In addition, the composition can further contain therapeutically effective matters.

The pharmaceutical formulation of the present invention can be administered orally, parenterally, for example subcutaneously, intravenously, intraperitoneally, or topically. The total daily dosage is in the range of 0.0001-100 mg/kg and can be divided into one to several portions and administered over several times. The level of administration dosage can be varied with the requirement of the subject patient, body weight, age, sex, medical condition, time and route of administration, evacuation rate, severity of the disease to be treated, and the like.

Further, the cosmetic composition according to the present containing oligolysine fusion protein as a main ingredient can be formulated in the form of creams, ointments, gels, lotions, etc. A person skilled in this art can easily determine the preferred formulation for a certain condition.

Still further, the lotion, gel, essence, cream and face lotion comprising the oligolysine fusion protein, for example, the oligolysine-SOD according to the present invention (in this invention, it is used together with “Lys-SOD”), oligolysine-CAT according to the present invention (in this invention, it is used together with “Lys-CAT”), as an active ingredient could be produced in all kinds of forms according to an usual producing process, and also it could be used as an anti-aging product, skin remedial agent by adding the oligolysine fusion protein into the base cosmetics.

For an example, generally, when producing the cream, the oligolysine-SOD fusion protein according to the present invention is added into an usual oil-in-water (O/W) type or water-in-oil (W/O) type cream base and perfume, chelate agent, pigment substances, anti-oxidant agent and antiseptic agent are additionally used. Meanwhile, in order to enhance physical properties, synthetic or natural substance such as protein, mineral, vitamin could be used.

The present inventors has observed that oligolysine-SOD fusion protein, oligolysine-CAT fusion protein, which are one of the oligolysine fusion protein, penetrates into the epidermis, dermis and further subcutaneous adipose tissue during their rat's skin penetration experiment. Accordingly, it is found that oligolysine fusion protein could be used as a main component for pharmaceutical composition and/or cosmetic composition.

The present invention is related to an oligolysine transport domain, which covalently combines with cargo molecule such like olygonucleotide, nucleic acid, peptide, protein, oligosaccharide or polysaccharide so as to penetrate into a cell, preferably composed of 3˜12 numbers of, and more preferably composed of 6˜9 numbers of lysine residues.

Also, the present invention is related to an oligolysine-cargo molecule complex, in which the oligolysine transport domain covalently combines with the olygonucleotide, nucleic acid, peptide, protein, oligosaccharide or polysaccharide so as to penetrate into a target cell, and exhibit the activities of the cargo molecule in the cell.

Still, the present invention is related to a fusion protein, in which the oligolysine transport domain, preferably composed of 3˜12 numbers of, and more preferably composed of 6˜9 numbers of lysine residues, covalently combines with cargo protein of carboxyl terminus so as to penetrate into a targetcell, and exhibit the activities of the cargo molecule in the cell.

Still further, the cargo molecule and/or cargo protein is selected form the group which is consisted of treatment molecule, prevention molecule and diagnosis molecule.

Still further, the present invention is related to an oligolysine fusion protein in which the cargo protein is green fluorescence protein or super oxide dismutase.

Still further, the present invention is related to a pharmaceutical composition which is composed of the oligolysine-cargo molecule complex that includes the oligolysine fusion protein in a pharmaceutically acceptable amount and also includes a carrier in a pharmaceutically acceptable amount.

Still further, the present invention is related to the oligolysine vector into which is inserted the oligonucleotide which has base sequence that is capable of expressing a plurality of, and preferably 3˜12, and more preferably 6˜9 lysine residues, for examples, sequence ID NO:1 and ID NO:3 or annealing products thereof, so as to produce the oligolysine-cargo molecule complex.

Still further, the present invention is related to an expression vector which produces the oligolysine-cargo complex as well as the oligolysine fusion that is fused with oligolysine transport domain on one side of the cargo molecule by inserting the cargo molecule cDNA on the oligolysine vector in order to express the oligolysine-cargo molecule.

Still further, the present invention is related to the expression vector which produces the oligolysine-cargo molecule complex as well as the olicolysine fusion protein which is characterized in that the cargo molecule is selected from treatment molecule, prophylactic molecule and diagnosis molecule.

Still further, the present invention is related to the expression vector which produces the oligolysine-cargo molecule including the oligolysine fusion protein characterized in that the cargo molecule is CAT or SOD.

Moreover, the present invention is related to a process for inducing the oligolysine-cargo molecule complex, which includes the steps of:

expressing the expression vector producing the oligolysine-cargo molecule complex from a microorganism;

purifying the expressed oligolysine-cargo molecule complex in denaturation condition or native condition by using 4˜6M urea;

adding the purified oligolysine-cargo molecule complex into the cell.

Further, the present invention is related to a process for inducing an oligolysine fusion protein into cell, which includes the steps of:

expressing the expression vector producing the oligolysine fusion protein from the microorganism;

purifying the expressed oligolysine fusion protein in denaturation condition or native condition by using 46M urea;

adding the purified oligolysine fusion protein to the cell.

Still further, the present invention is related to a method for preventing and treating diseases using the oligolysine-cargo molecule complex or expression vector producing the same.

Still further, the present invention is related to the method for preventing and treating SOD related diseases, for examples, glomerulonephritis, angiitis, autoimmune disease, apoplexy, mycocardial infarction, dysrhythmia, angina pectoris, idiopathic hemocromatosis, disease occurred from radiation treatment, progeria, disease-related aging, sickle-cell anemia, malaria, pulmonary emphysema, myocardiopathy, autoimmune nephrotic syndrome, Betelnut-related oral cancer, hyperbaric oxygen disease, Alzheimer's disease, Perkinson's disease and cataract etc.

Also, the present invention is related to a cosmetic composition comprising the oligolysine-cargo molecule complex including the oligolysine fusion protein as an active component.

Moreover, the present invention is related to the cosmetic composition comprising the oligolysine fusion protein as the active component and also having functions of removing active oxygen groups.

Furthermore, the present invention is related to the cosmetic composition characterized in that it could be manufactured in facing lotion type, gel type, water-soluble liquid type, oil-in-water (O/W) type and water-in-oil (W/O) type.

Reference will now be made in detail to the preferred embodiments of the present invention, examples of which are illustrated in the accompanying drawings.

It is to be understood that both the foregoing general description and the following detailed description of the present invention are exemplary and explanatory and are intended to provide further explanation of the invention as claimed.

Materials

The examiners have purchased materials for experiments produced by certain companies in the following.

Restriction enzyme and T4 DNA ligase is produced by Promega (USA) and Pfu polymerase is produced by Stratagene (USA). Oligonucleotide used in experiment is prepared from custom primer of Gibco BRL (USA). IPTG is produced by Novagen (USA), and Ni-nitrilotriactic acid speharose superflow is produced by Qiagen (Germany). Column P10 which is used to remove salinity of purified protein is produced by Amersham Pharmacia (USA). Reagent for preparing SDS produced by Sigma (USA), and kit is produced by BIO-RAD. Culture solutions such as DMEM (Dulbecco's Modified Eagle Medium), FBS (Fetal bovine serum), NCS (Newborn calf serum), which are used for culturing cell, are produced by GIBCO BRL, and Olympus epifluorescence microscope is used to observe whether GFP protein is fluorescent or not and distribution of the GFP protein within cell. All reagents besides above materials are A grade substances, and all of then are to be regarded that they are produced by Sigma if not particularly mentioned of the name of manufacturing company.

### EXAMPLE 1

### Preparation of Oligolysin Fusion Protein Expression Vector

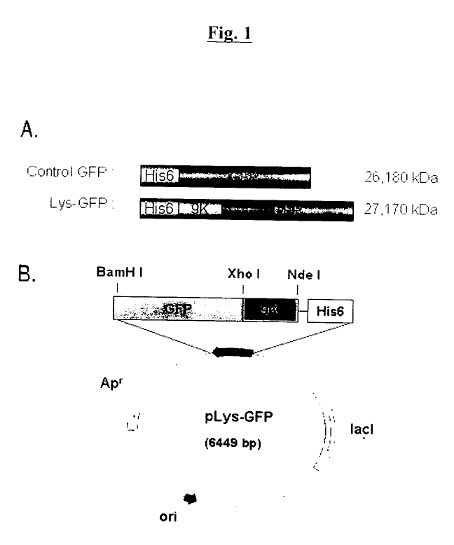

In order to develop a technology to introduce a protein or peptide having a function into a cell an expression vector for a fusion protein, i.e., pLys, being capable of delivering a target protein into a cell, was prepared. To make it possible to easily analyse the ability of nine lysine residues to deliver a protein into a cell, a pLys-GFP fusion protein vector which is a vector capable of expressing a Lys-GFP fusion protein was prepared by selecting GFP as a target protein and inserting a DNA fragment corresponding to a base sequence of GFP into pLys vector (FIG. 1B). To over-express GFP which is a control protein for the oligolysine-GFP (Lys-GFP) fusion protein, a pGFP expression vector, which is the same as pLys-GFP except that it does not comprise nine lysine residues, was developed.

The oligonucleotide which have double strand was inserted to pET-15b vector (Invitrogen, Carlsbad, Calif.) cleaved with Nde I and Xho I restriction enzyme, said oligonucleotide being produced to anneal oligonucleotides having base sequence expressing nine lysine, i.e. to anneal both forward primer:5′-TAAGAAGAAAAAGAAAAAGAAAAAGAAGC-3′; reverse primer: 5′-TCGAGC TTCTTTTTCTTTTTCTTTTTCTTCT-3′ at 100° C. for 5 min, and the pLys which is vector expressing nine lysine was produced. Therefore, newly provided vector was designed so that six histidine, nine lysine residues and a target protein were expressed as fusion protein form. (FIG. 1A) Then, after selecting Green Fluoresscence Protein (“GFP” designated in this specification) as target protein, two kinds of olgonucleotide was synthesized based on cDNA's base sequence of GFP. The forward primer had Xho I restriction enzyme cleavage site as 5′-CTCGAGGTGAGCAAGGGCGAGGAGCTG-3′ and the sequence of reverse primer had BamH I restriction enzyme cleavage site as 5′-GGATCCTTACTTGTACAGCTCGTCCATGCCGAG-3′.

To using the synthesized oligonucleotide, DNA fragment having GFP base sequence was prepared from pEGFP-C2 (Clontech) conducting polymerase chain reaction (PCR) as follows. The PCR was conducted in thermal cycler (Perkin-Elmer, model 9600), reaction mixture was put in 50, siliconized reaction tube and was heated at 94° C. for 3 min. PCR was induced extension reaction 30 times at 94° C. for 30 sec, denaturation at 51° C. for 45 sec, annealing at 70° C. for 1 min 30 sec, and final extension at 72° C. for 10 min, at 20° C. for 5 min.

After conducting PCR, products were separated with agarose gel electrophoresis and inserted in pGEM-T vector (Promega, USA). Then, the vector was transformed on competent cells and the plasmid was separated from transformed bacteria using alkaline lysis method (Sambrook et al, 1989) pGEM-T vector including GFP cDNA was cleaved with Xho I and BamH I, and then inserted in pET-15b and pLys expression vector. Accordingly, obtained vector was designated pGFP and pLys-GFP individually. (FIG. 1)

### EXAMPLE 2

### Expression and Confirmation of a Fusion Protein

Escherichia coli cell, induced with IPTG to overexpress a fusion protein, was disintergrated by ultrasonication at 4° C., done with centrifugal separation and the protein presented in supernatant of the separated solution was separated using 12% polyacrylamide gel eletrophoresis.

After E. coli BL21 (DE3) transformed with pGFP and pLys-GFP was selected, colony was inoculated into 50 ml of LB culture medium, IPTG (0.5 mM) was added to the culture medium overexpression of a fusion protein was induced and the cell extract was prepared. Over-expressin of recombinant GFP and Lys-GFP fusion protein was induced. Over-expressed GFP and Lys-GFP fusion protein presented in the cell extract was confirmed with SDS (sodium dodecyl sulfate) polyacrylamide gel eletrophoresis and Western Blot analysis.

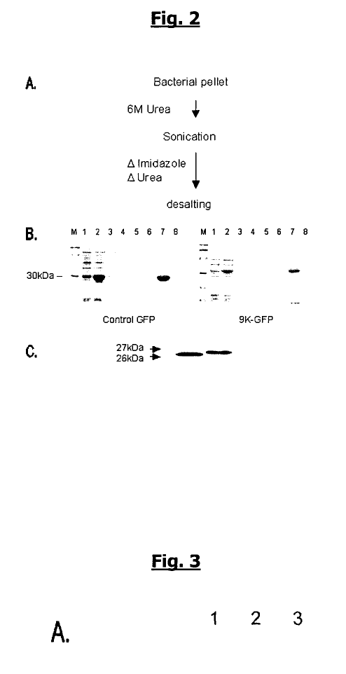

FIG. 2-B show the protein band stained with Coomassie brilliant blue. The lane 2 of gel in which a fusion protein of very high concentration is over-expressed, was shown as compared with lane 1 (pET-15b vector).

### EXAMPLE 3

### Purification of Fusion Protein

In order to compare permeation capability in cells mutually, the fusion protein was purified on denaturing condition and native condition individually. In order to obtain partially denatured fusion protein, lysate which is obtained to break cell by ultrasonication in solution including 6M urea was purified through Ni-NTA column (nitrilotriacetic acid sepharose column) and the PD-10 column chromatograph was used to remove salt.(FIG. 2A)

Because the fusion protein is comprised of six histidine on N-terminus, immobilized metal-chelate affinity chromatography was used, so that a fusion protein was purified purely (purity>90%) (FIG. 2B). The product obtained by such process was confirmed, that the molecular weight of Lys-GFP fusion protain and control GFP was 27 kDa, 26 kDa individually as shown FIG. 2C using Western Blot with GFP polyclonal antibody.

In this study, we intended to use the fusion protein having nine lysine residues overexpressed according to following methods, and purified on native condition or denaturation condition, in permeation experiment of a target cell. The E. coli BL21 transformed with pLys-GFP vector was added to LB culture medium including ampicillin, was stired on 37° C. at 250 rpm and was cultivated. When bacteria concenturation in media solution designated O.D600=0.6-1.0, IPTG was added to the culture medium so that the final concenturation was amount to 0.5 mM and then was cultivated further for 3 hour. After the cultivated cell was collected by centrifugal separation, 5 ml binding buffer solution (5 mM of imidazole, 0.5M of NaCl, 20 mM of Tris-HCl, pH 7.9) was added and treated by sonication. The recombinant Lys-GFP fusion protein was purified under two condition, i.e. denaturing condition or native condition individually. 10 g of bacterial pellet was added in 40 ml of buffer solution A (6M of urea, 20 mM of HEPES, pH 8.0, 100 mM NaCl), was disintegrated by ultrasonication, and then was conducted twice to centrifuge (16,500 rpm×30 min, 40,000×30 min, 4° C.) so that insoluble cell debris was removed.

Supernatant of the separated solution obtained by centrifugal separation was loaded immediately to 2.5 ml of nitrilotriacetic acid sepharose column (Quagen, USA) buffered by buffer solution B (6M of urea, 20 mM of HEPES, pH 8.0, 100 mM NaCl, 20 mM of imidazole), was washed with binding buffer solution of 10 times volume and washing buffer (60 mM of imidazole, 0.5M of NaCl, 20 mM of Tris-HCl, pH 7.9) of 6 times volume and then the Lys-GFP fusion protein was eluted with elution buffer solution (1M of imidazole, 0.5M of Nacl, 20 mM of Tris-HCl, pH 7.9). Then, a fraction in which Lys-GFP fusion protein is included, was collected and a salt included in the fraction was removed conducting PD-10 column chromatography, wherin PD-10 column comprising Sephadex G-25M.

The protein concentration of the fraction was determined with Bradford method using calf serum albumin (Bradford, 1976).

### EXAMPLE 4

### Transducing Experiment of Fusion Protein in Cell

Firstly, transducing efficiencies in HeLa cell of Lys-GFP fusion protein purified in denaturing condition and native condition are measured.

HeLa cell is cultured in DMEM (Dulbecco's Modified Eagle's Medium) including 20 nM HEPES/NaOH (pH 7.4), 5 nM NaHCO₃, 10% of fetel bovine serum (FBS) and antibiotic agent (100 μl/ml of streptomycin, 100U/ml of penicillin) at 37° C. with which is provided of 95% of air and 5% of C02. In case of PC12 cell, as a longitudinal culture medium, 5% of FBS and 10% of horse serum is added into RPMI 1640. Also, 20 nM HEPES/NaOH (pH 7.4), 5 nM NaHCO₃, antibiotic agent (10,000 μg/ml of streptomycin, 10,000U/ml of penicillin) is added into each culture medium at 37° C. with which is provided of 95% of air and 5% of CO₂.

To observe the Lys-GFP fusion protein penetrating into the cell, HeLa cells are cultured in 6-well plates for 4-6 hrs which then are shifted with fresh culture media comprising 1 ml of FBS and treated a recombinant Lys-GFP in several concentration in the culture media. After 1 hr, the cell is treated with trypsin-EDTA (Gibco BRL) and then sufficient washed with PBS. After the cell is disintegrated, the amount of Lys-GFP fusion protein penetrated into the cell is analyzed in Western blotting method and measured with immuofluorescence microscope.

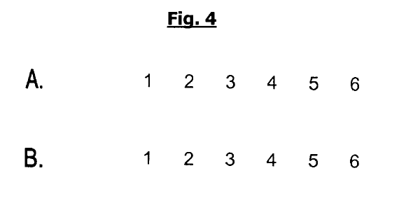

The respective Lys-GFP fusion protein purified in denaturation condition and native condition is treated in cell culture solution in 0.5 μM for 2 hrs, and as a result, it is observed by Western blotting that the denatured Lys-GFP fusion protein is transduced into the cell in high rate compared with the Lys-GFP fusion protein purified in native condition, and control-GFP used as control protein could not penetrate into cell membrane (FIG. 3). This result reflects the fact that when the protein is transduced into the cell, denatured Lys-GFP fusion protein shows higher penetration efficiency rather than the native Lys-GFP. To examine the above phenomenon in energetic manner, the reason why the denatured Lys-GFP fusion protein shows higher penetration efficiency is that unfolded protein owing to denaturization is much easier to penetrate into the cell membrane rather than the protein having tertiary or quaternary structure.

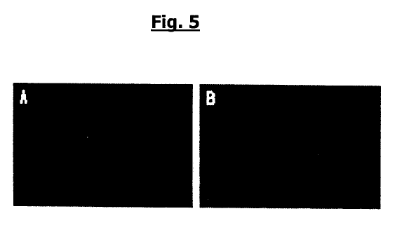

The change of HeLa cell penetration of denatured Lys-GFP fusion protein according to time and concentration is observed. As shown in FIG. 4, denatured Lys-GFP fusion protein is transduced into the HeLa cell depending on the time and concentraition. The concentration of denatured Lys-GFP fusion protein is gradually increased from 0.1 μM to 1.5 μM for 2 hrs, and as the result, as shown in FIG. 4-A, the penetration quantities are observed to be increased as the concentration is increased. Also, as the result of observing the penetration rate of 0.5 μM of Lys-GFP fusion protein into the cell by time, the highest rate is shown when the fusion protein is penetrated into the cell membrane after 5 mins elapsed from the administration of the fusion protein and treated for 1 hr, and the same rate is observed when treated the fusion protein for 6 hrs (FIG. 4B).

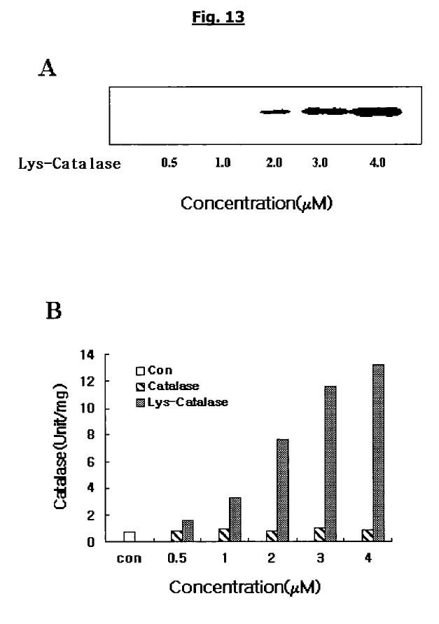

Also, to observe the transducing efficiency of the Lys-CAT into the cell by experimenting in a similar way as above, 0.5˜4 μM of recombinant protein is treated in PC12, and likewise, it is resulted in that the penetrated protein amount is increased in proportion to the concentration (FIG. 13A), and activity is increased for about 13 times much than the cell that is not being treated of the protein. The cell treated of catalase showed the similar value with that of the control cell (FIG. 13B).

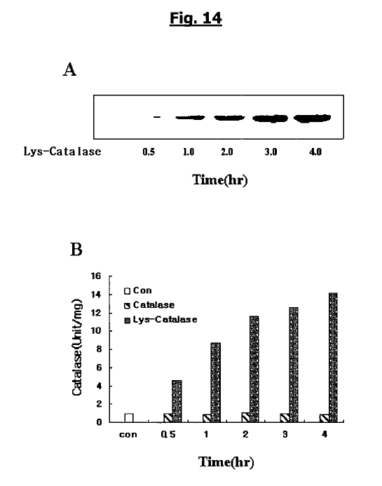

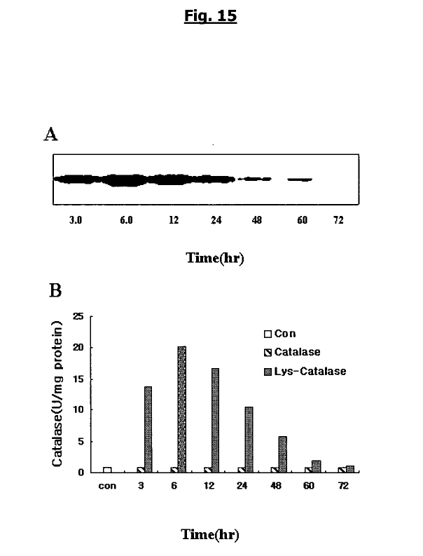

The penetration efficiency into the cell by time is observed by experimenting in the way that 4 μM of Lys-catalase is treated in PC12 cell for 0.5˜4 hrs. As the result, it is observed that the recombinant protein is increased as the time passes (FIG. 14A), and after 30 mins elapsed form treatment, the activity of the catalase is increased for about 4.5 times, and after 4 hrs, catalase is increased for 14.5 times (FIG. 14B). However, the increase of the catalase activity in the cell is not observed when the catalase used as the control protein is treated in the cell.

### EXAMPLE 5

### Western Blotting Analysis

A GEP and a Lys-GFP protein within a disintegrated cellular solution is separated through a 12% SDS polyacrylamide gel electrophoresis, and then a protein in the gel was transferred to a nitrocellulose membrane (Amersham, UK). A Lys-SOD and Lys-CAT protein were electrically transferred to PVDF (polyvinylidene fluoride) membrane (Millipore).

The Nitrocellulose membrane to which the GFP or Lys-GFP protein was transferred was blotted with a 5% dry milk and a 3% Tween 20, and thereafter treated with a polyclonal antibody (Clontech, USA, 1:1,000) against the GFP for an hour. After washing, the membrane reacted with a goat anti-rabbit IgG antibody (Sigma, 1:10,000 dilution) covalently combined with an alkaline phosphatase for an hour. Finally, an alkaline phosphatase conjugate substrate kit (Bio-Rad, USA) was treated, so that a protein band reacting with the GFP polyclonal antibody was identified.

The PVDF membrane to which the Lys-SOD and Lys-CAT protein were transferred was treated with a 5% non-fat milk solution, and treated with a histidine antibody against a human SOD and a human catalase for one hour. After washing, the membrane reacted with a goat anti-rabbit IgG antibody (Seoulin, 1:10,000 dilution) for one hour. Finally, an enhanced chemiluminescent substrate kit (Amersham; ECL) was treated, so that a protein band reacting with the human catalase antibody was identified.

### EXAMPLE 6

### Immunofluorescence Microscopic Analysis

It is important how much percentage of cells are transduced when a fusion protein is needed to be transduced to a target cell. Such a protein transduction technology is usable only if the fusion protein penetrated into inside the cell maintains its activity. Therefore, the present experiment analyzed the percentage of cells when the oligolysine fusion protein was transduced or whether the transferred fusion protein preserves the activity. Further, the present experiment observed a degree of fluorescence of the GPF in a HeLa cell, which treated the denatured oligolysine fusion protein Lys-GFP, by using a fluorescence microscope.

The HeLa cell is respectively cultured in 24 well plates into which a cover glass had already been added, so as to become 5×10⁵. Before treating the Lys-GFP fusion protein in the HeLa cell, the culture medium was thrown away and washed with a PBS (pH 7.4). After that, a new complete medium of 1 ml wherein the FBS was added to the respective wells was added, and the Lys-GFP fusion protein was treated by time and by density. After the medium was thrown away with the lapse of 2 hours and washed one or two times with PBS, the cell was treated with a trypsin-EDTA (Gibco BRL) and washed one or two times with the PBS. In order to fix the cell, a 3.7% formaldehyde was used. The cover glass was held with tweezers, soaked in a secondarily distilled water for a little while, taken out, and sufficiently dried at room temperature. A mounting solution which is a PBS containing a 90% glycerol and a 0.1% phenylenediamine was treated in a slide glass, and then the cover glass which was previously prepared was put on, whereby distribution of the fluorescence of protein within the cell was analyzed and whether the fluorescent exists within the cell was observed by using an olympus epifluorescence microscope (Eric et al., 1997).

The denatured Lys-GPF fusion protein was transduced to the HeLa cell of almost 100%. It is found that the activity of the GFP protein within a cytoplasm was recovered after penetration of the HeLa cell (see FIG. 5). The degree of fluorescence within the HeLa cell which treated the denaturalized Lys-GFP fusion protein was found to be increased depending on density, similar to Western blot data. Meanwhile, when the Lys-GFP fusion protein was treated in a low density, most of protein was accumulated in the cytoplasm. As the density was gradually increased, the protein was accumulated in the cell nucleus and the cytoplasm. The degree of fluorescence in a cell to which the fusion protein was not given is so low that the degree is similar to that of a control group. These results showed that the Lys-GFP fusion protein was successfully penetrated into almost the cells and the penetrated protein has exhibited a complete activity. In addition, the denaturalized Lys-GFP fusion protein recovered its original condition in structure within the HeLa cell.

### EXAMPLE 7

### Preparation of Vector Expressing Lys-GFP Fusion Proteins Consisting of 5-9 Lysine Residues and Production of the Fusion Protein and Purification Thereof

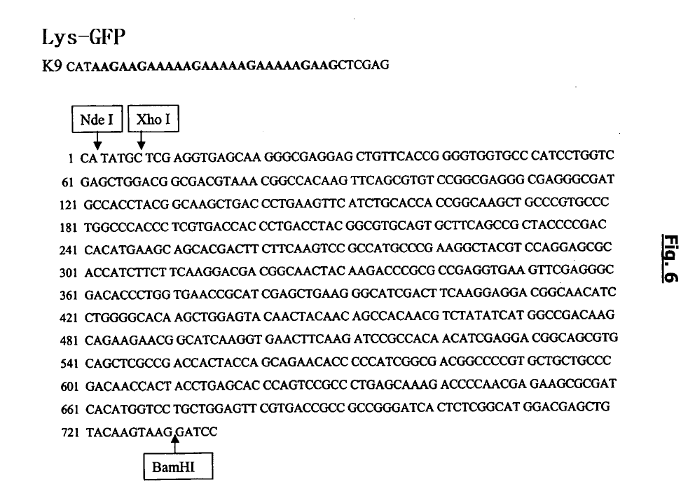

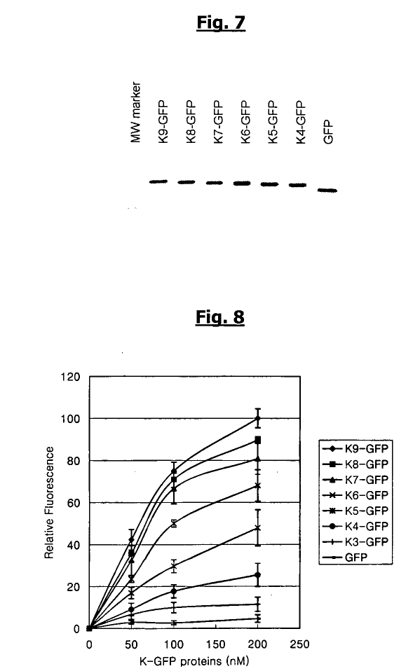

The vector expressing oligolysine (used together with “9K”, “Lys” in the specification and figures)-GFP fusion proteins was prepared by synthesizing an oligonucleotide encoding from 3 to 9 lysine and respectively ligating it to pET15b-GFP cleaved with Nde I and Xho I, and the sequence of bases inserted was identified by sequencing (FIG. 6). These fusion proteins were fused in order from amino acid terminal to histidine tag (his tag)-olgolysine (9K)-GFP, wherein the GFP used was proteins derived from Aequorea victoria. As a control, Tat-GFP fusion proteins, cell permeability of which was proved in S2 cells, were used.

In order to express said oligolysine-GFP fusion proteins excessively, said pET-oligolysine-GFP expression vector was induced with said IPTG to express excessively, and E. coli cell treated as above was disintegrated by ultrasonic treatment in a solution containing 6M Urea, and then purified.

The plasmid prepared as above was transformed into E. coli BL21, expressing fusion protein attached with his tag, and then the cell was broken at denaturing condition as the method shown in Novagen, purified with Ni column to be almost pure, more than 95% of purity (FIG. 7). The eluted protein was well mixed with equivalent volume of glycerol, and then stored in deep freezer at −70° C. The concentration of purified protein was measured with Bradford assay, and identified by SDS-PAGE.

### EXAMPLE 8

### Comparison of Cell Penetrating Activity of Lys-GFP Fusion Proteins Using Fluorometer

The measurement of cell penetrating activity of Drosophila S2 cell culture and PTD-GFP fusion proteins followed the method of prior arts (Han, 1996; Han et al, 2000). S2 cell requiring splitting and four-fold volume of M3 culture medium (Sigma) were well mixed, and then cell suspension was transferred in 0.5 ml to each well of 24 well culture plate. A several μl (50 nM, 100 nM, 200 nM) of purified fusion proteins were added to 2 wells per concentration of culture medium, and well mixed. The period from transferring of cell to culture plate, to adding protein made no difference from immediately to about 12 hrs. Generally, the proteins were treated after 2 hrs, and then again after 1 hr., the culture medium was changed to 100 μl of trypsin free of EDTA (Gibco), followed by culturing for 15 mins. at 25° C. After being added 400 μl of PBS, the culture medium was transferred to microfuge tube by using micropipette, and at this time, cell suspensions of 2 wells, having equivalent concentration, were combined. After centrifuging for 5 mins. at 2,000 rpm, precipitated cells were washed 3 times with 500 μl of PBS. Quantitative analysis for GFP fusion proteins was carried out with fluorometer (excitation: 488 nm, emission: 507 nm).

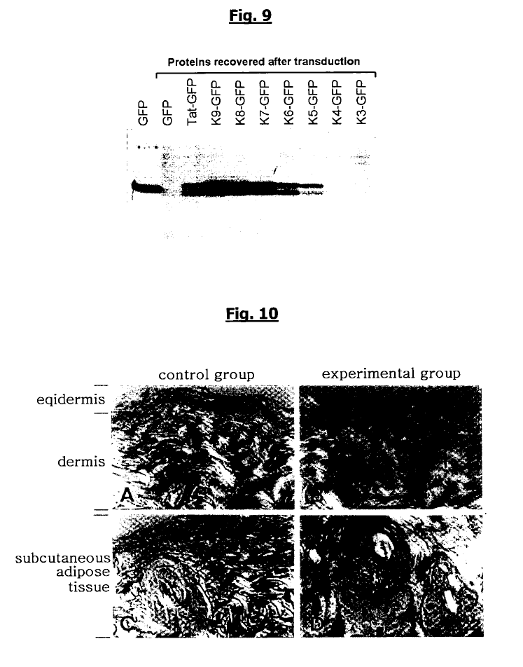

The penetration efficiency of Lys-GFP fusion proteins was proportionate to the concentration of the proteins, and furthermore, if residues of lysine were 3-9 ea., the efficiency went up proportionately as the number of residues increased. (FIG. 8).

### EXAMPLE 9

### Western Blotting

In order to compare the penetration efficiency and stability in cell, of Lys-GFP fusion proteins, western blotting was executed.

Penetration of fusion proteins into cell was executed as the method mentioned above, except ten-fold increase of the scale. That is, 60 mm of culture dish and 5 ml of cell suspension were used, and fusion proteins were added to be 300 nM of the final concentration. 1 hr after treating proteins, the cell was treated with 1 ml of trypsin, and washed with PBS several times, and then broken by 20 μl of CLR (cell lysis reagent, Promega). The resulting product was mixed with 5 μl of 5× loading buffer, and the mixture was boiled for 5 mins, being centrifuged for 10 mins. Concentration of proteins was measured with Bradford assay, and then volume of each cell extract was adjusted to be equivalent amount of protein, and then supernatant was loaded to 10% SDS polyacrylamide gel, and electrophoresis was executed. The separated proteins were transferred to nitrocellulose membrane (Amersham, UK), and then blocked with PBS containing 5% BSA. The proteins were treated by polyclonal anti-GFP antibody for 1 hr at atmospheric temperature and washed, and then reacted with horseradish peroxidase-conjugated goat anti-mouse IgG antibody for 1 hr at atmospheric temperature. Position of GFP proteins was found by ECL kit (Amersham) using the method provided by Amersham.

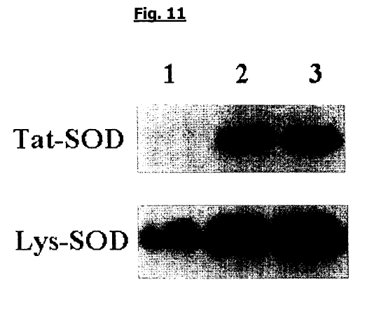

GFP proteins didn't have permeability at all, from the fact that no protein band was observed in western blotting. On the contrary, all oligo K-GFP fusion proteins formed protein bands, at predicted molecular spot, in pattern similar to the result measured with fluorometer (FIG. 9). Furthermore, we have found that when the number of lysine was more than 6 ea. and less than 9 ea., the penetrating efficiency was high, compared with that of Tat-GFP.

### EXAMPLE 10

### Preparation of Vector Expressing Lys-CAT, Lys-SOD and Transformation

Two kinds of oligonucleotides corresponding to lysine residues 9 ea. were inserted by ligation to pET15b cleaved with Nde I-Xho I restriction enzyme.

Subsequently, two kinds of oligonucleotides were synthesized on the basis of the sequence of cDNA of human kidney catalase. Forward primer of catalase was 5′-ctcgagatggctgacagccgggatcccgcc-3′, containing Xho I restriction site, and the sequence of backward primer was 5′-ggatcctcacagatttgccrrctcccttgc-3′, containing BamH I restriction site.

On the basis of the sequence of cDNA of humane Cu, Zn-SOD, two kinds of oligonucelotides were synthesized. Forward primer was 5′-CTCGAGGCGACGAAGGCCGTGTGCGTG-3′, containing Xho I restriction site, and the sequence of backward primer was 5′-GGATCCTTATTGGGCGATCCCAATTAC-3′, containing BamH I restriction site.

From the oligonucleotides synthesized as above, PCR was executed as the method of Example 1, to produce pLys-CAT, pLys-SOD vector.

### EXAMPLE 11

### Expression, Identification and Purification of Lys-CAT, Lys-SOD Fusion Proteins

Expression, identification and purification of Lys-CAT, Lys-SOD fusion proteins was executed according to the method of said Example 2 and 3.

### EXAMPLE 12

### Measurement of Enzyme Activity of Lys-CAT Fusion Proteins

Activity of catalase was measured by observing with spectrophotometer, the degree of hydrogen peroxide degradation by catalase/hydrogen peroxide reaction, according to the method of Aebi (1980).

In standard analytical process, the degradation velocity of hydrogen peroxide was measured at 240 nm by adjusting the final concentration of hydrogen peroxide, in 50 mM of potassium phosphate buffer: pH 7.0 at 25° C., to 10 mM. 1 unit was defined as the amount of catalase dissolving hydrogen peroxide for 1 min under the conditions mentioned above.

### EXAMPLE 13

### Stability of Fusion Proteins Penetrated into Cells

In order to measure the stability of fusion proteins penetrated into cells, cells were cultured at Swell plate, and then culture medium was changed to 1 ml of fresh culture medium not containing serum. 2 uM Tat-catalase and 4 uM Lys-catalase were treated hourly in culture medium, and then the cells were sufficiently washed with trypsin-EDTA (Gibco BRL), PBS. Furthermore, the cells were collected, and then the amount of catalase in the cells was analyzed by western blotting, and the activity was measured.

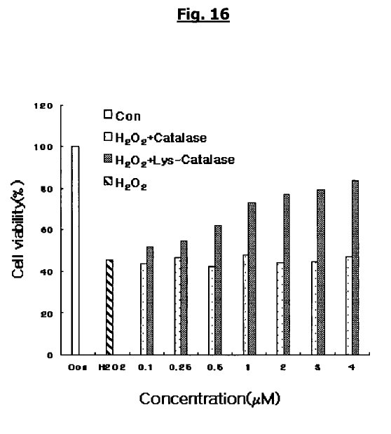

PC12 cells were treated with 4 uM of Lys-catalase, and then the maximum penetrating amount and stability was examined hourly with western blotting. As shown in FIG. 15A, proteins penetrated in largest amount to 6 hrs, and as time went by, Lys-CAT penetrated in cells was degraded and reduced, but the degradation velocity was very low. Once penetrated into cell, penetrated fusion protein was almost never degraded till 24 hrs, and existed in cell. Furthermore, activity of catalase also showed stability in proportion to the amount of protein penetrated into cell, showing stability about two-fold of that of control to 60 hrs.(FIG. 15B).

Such results means that the oligolysine transducing domain-coupled fusion proteins were well penetrated into cells, and the stability in cells also keeps high, therefore, showing the possibility as protein therapeutic agents much higher.

### EXAMPLE 14

### Measurement of Survival Ability of Cell by Hydrogen Peroxide

PC12 cells were cultured for 24 hrs. at 24-well plate, and then treated with recombinant protein, according to each concentration (0.1-4 uM), in fresh culture medium not containing serum, allowing proteins to penetrate into cells for 6 hrs. Furthermore, fresh medium and hydrogen peroxide were added to cleanly washed cell, to be 0.5 uM, which were treated for 3 hrs., and the medium was removed, and then MIT (1 mg/id phenol red free medium) solution were added in 0.5 ml to 24-well. After 3 hr culture, NM solution was removed, and 100% isopropanol was added in 0.5 ml and then, with ELISA reader, absorbance was measured at 570 nm, therefore, the survival ability of cells by catalase penetrated into cells was analyzed.

The Catalase is known from long time ago, as an enzyme finally dissolving hydrogen peroxide into water and oxygen in biological body. When cell toxicity was induced by hydrogen peroxide, Lys-catalase penetrated into cells increased the survival ability of cells more than 40% of that of cells not treated with recombinant proteins (4 μM addition), and the control catalase didn't show increase of cell survival rate (FIG. 16).

From these results, it is considered that Lys-CAT penetrated into cells has enough ability to dissolve hydrogen peroxide, which is applicable to purpose of treating all sorts of diseases.

### EXAMPLE 15

### Penetration of Lys-SOD, Lys-CAT into Animal Skin Cells

As experimental animals, about 200 g of body weight, male SD rats were used. For experimental animals, control (SOD application) 10 heads and experimental group (Lys-SOD application) 15 heads were used respectively.

Each experimental animal was kept fasting for 24 hrs, and then induced to general anesthesia by 3% isoflurane with the gas mixed with nitrogen and oxygen in the ratio of 7:3 and then, with anesthesia state kept by 2.5% isoflurane with the same gas, SOD and Lys-SOD were respectively applied in 0.3 mg to abdominal skin.

Normal group and experimental group were put under general anesthesia by injecting of ketamine 30 mg/kg to abdominal cavity 2 hrs. after the application, being butchered, and then the skin of application site was cut, and then 10 μm frozen tissue slice was prepared according to typical process

Prepared tissue slice was fixed for 10 mins. with 4% paraformaldehyde, being washed 3 times for 10 mins. with 0.1 M PBS, pH 7.4, to remove fixing solution in the tissue. Then, to prevent nonspecific immunoreactivity, reaction was executed for 1 hr at room temperature, with the solution which was 0.3% Triton X-100, 10% normal goat serum diluted with 0.1M PBS (PH 7.4). Thereafter, rabbit anti-histidine Ig (9 rabbit anti-histidine IgG), primary antibody, was diluted in the ratio of 1:500, and reacted with the tissue for 24 hrs. at room temperature. Then, the primary antibody in tissue slice was removed by washing three times for 10 mins., with 0.1M PBS, and then IgG (biotinylated goat anti-rabbit IgG) (Vector Laboratories, USA), secondary antibody, was diluted in the ratio of 1:200, with 1M PBS, and reacted with the tissue for 1 hr. at room temperature. The tissue, in which the reaction was completed, was washed 3 times for 10 mins. with 0.1M PBS, and then peroxidase-conjugated streptavidin (Vector Laboratories, USA) was diluted in the ratio of 1:300 (in 0.1M PBS), reacted with the tissue for 1 hr at room temperature.

The tissue slice, in which the immunoreaction was completed, was colored for 3 mins. with substrate solution (40 mg diaminobenzidine/0.045% H₂O₂ in 100 ml PBS). To avoid excessive coloring, degree of coloring was continuously checked under optical microscope. The tissue slice, in which coloring was completed, was attached to slide, and then washed several times with distilled water and 0.1M PBS, and permanent specimen was prepared by way of dehydration with typical process.

With control CAT and Lys-CAT, skin penetration experiment was executed according to the same process.

The result of examples was that, in case of SOD application group, histidine immunoreaction wasn't almost observed (FIGS. 10A and C), but, in case of Lys-SOD application group, strong histidine reaction was observed all over the layers of epidermis layer, dermis layer and hypodermis tissue (FIGS. 10B and D). Histidine immunoreaction observed in Lys-SOD application group was mainly observed in nucleus, and cells, in which such immunoreaction was observed, were uniformly observed, epidermis cell, firoblast and follicle and their neighboring cells, etc.

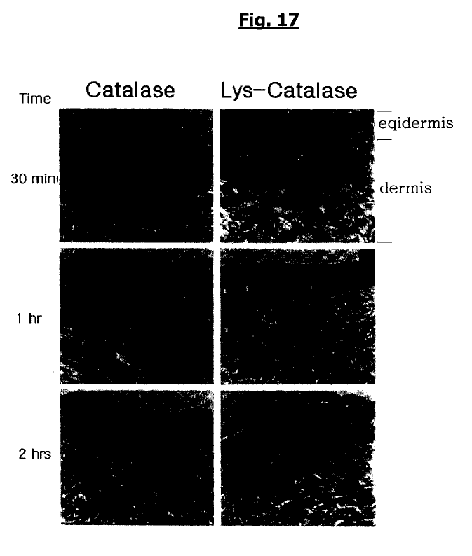

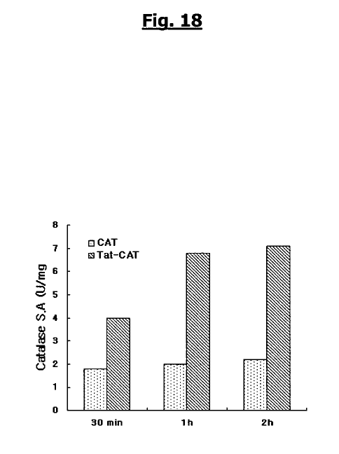

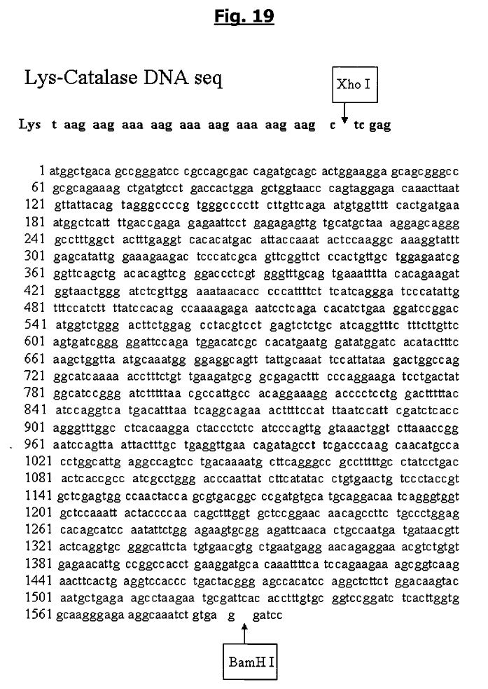

The result of the study was that, in case of catalase control, histidine immunoreaction wasn't almost observed (FIG. 17A, C, E), but, in case of Lys-CAT experimental group, strong histidine immunoreaction was observed over epidermis layer, dermis layer (FIG. 17B, D, F). Histidine immunoreaction observed in Lys-CAT experimental group was chiefly observed in nucleus, and cells, in which such immunoreaction was observed, was uniformly observed, epidermis cell, firoblast and follicle and their neighboring cells, etc. Furthermore, it was observed that activity of Lys-CAT penetrated into skin tissue increased about twofold at 30 mins., and, as time went by, about four-fold (FIG. 18).

### EXAMPLE 15

### Comparative Experiment of Efficiency of Penetration into HeLa Cell of Lys-SOD and Tat-SOD

In order to compare efficiency of penetration into HeLa cell of Lys-SOD and Tat-SOD, experiment was executed as the method of experiment 4 and 5. To lane 1, 2, 3 of FIG. 11, Lys-SOD, Tat-SOD in 0.2 μM, 0.5 μM, 1 μM, respectively, were added, and degree of penetration into HeLa cell was identified with western blotting.

As a result, as shown in FIG. 11, it was indicated that the cell penetration efficiency of Lys-SOD was even higher than that of Tat-SOD.

### INDUSTRIAL APPLICABILITY

As fully explained and demonstrated, with regard to oligolysine transporting domain and oligolysine-cargo molecular complex, oligolysine fusion protein, vector thereof, etc. as described in the invention, we have found that purified oligolysine-cargo molecule complex was efficiently transmitted into cell, and the activity of cargo molecules transmitted was retained, and also, in the experiment of skin cell penetration, the molecules penetrated into skin. Therefore, the oligolysine-transporting domain will be applied as an efficient transmitter and a method of introducing, of biologically active material into cell.

Although embodiments have been illustrated and described, various modifications may be made without departing from the spirit and scope of the present invention, which is intended to be limited solely by the appended claims.

What is claimed is:

1. An oligolysine transport domain covalently bonded to a cargo molecule having a biological activity that is non-penetrating or hard to penetrate into a cell or a cell nucleus, thereby allowing the cargo molecule to penetrate into the cell or the cell nucleus.

2. The oligolysine transport domain as claimed in claim 1, wherein the cargo molecule is an oligonucleotide, peptide, protein, oligosaccharide, or polysaccharide.

3. The oligolysine transport domain as claimed in claim 1 or 2, wherein the oligolysine transport domain comprises 3 to 12 lysine residues.

4. The oligolysine transport domain as claimed in claim 3, wherein the oligolysine transport domain comprises 6 to 9 lysine residues.

5. An oligolysine-cargo molecule complex comprising the oligolysine transport domain covalently bonded to the cargo molecule according to any one of claims 1 to 4, thereby the oligolysine-cargo molecule complex being capable of penetrating into a cell or a cell nucleus and having an activity in the cell or the cell nucleus.

6. An oligolysine fused protein comprising the oligolysine transport domain according to any one of claims 1 to 4 at an amino terminus thereof and a cargo molecule at a carboxyl terminus thereof, the oligolysine transport domain being covalently bonded to the cargo molecule, thereby the oligolysine fused protein being capable of penetrating into a cell or cell nucleus and having an activity in the cell or the cell nucleus.

7. The oligolysine fused protein as claimed in claim 6, wherein the cargo molecule is selected from a therapeutic molecule, a preventive molecule, or a diagnostic molecule.

8. The oligolysine fused protein as claimed in claim 6, wherein the cargo molecule is catalase (CAT) or superoxide dismutase (SOD).

9. A pharmaceutical composition comprising a pharmaceutically effective amount of the oligolysine-cargo molecule complex according to claim 5, and a pharmaceutically acceptable carrier.

10. A pharmaceutical composition comprising a pharmaceutically effective amount of the oligolysine fused protein according to any one of claims 6 to 8, and a pharmaceutically acceptable carrier.

11. An oligolysine vector with an oligonucleotide having a base sequence to express the oligolysine transport domain according to any one of claims 1 to 4, thereby the oligolysine vector being capable of producing the oligolysine-cargo molecule complex according to claim 5 or the oligolysine fused protein according to any one of claims 6 to 8.

12. The oligolysine vector as claimed in claim 11, wherein the oligonucleotide having a base sequence to express the oligolysine transport domain is sequence ID NO:1, sequence ID NO:3, or an annealing product therof.

13. An expression vector comprising a cargo molecule cDNA inserted into the oligolysine vector according to claim 11 or 12 in order to express the oligolysine-cargo molecule complex according to claim 5, thereby the expression vector producing the oligolysine-cargo molecule complex comprising an oligolysine transport domain at the carboxyl terminus of a cargo protein.

14. An expression vector comprising a cargo molecule cDNA inserted into the oligolysine vector according to claim 11 or 12 in order to express the oligolysine fused protein according to any one of claims 6 to 8, thereby the expression vector producing the oligolysine fused protein comprising an oligolysine transport domain at the carboxyl terminus of a cargo protein.

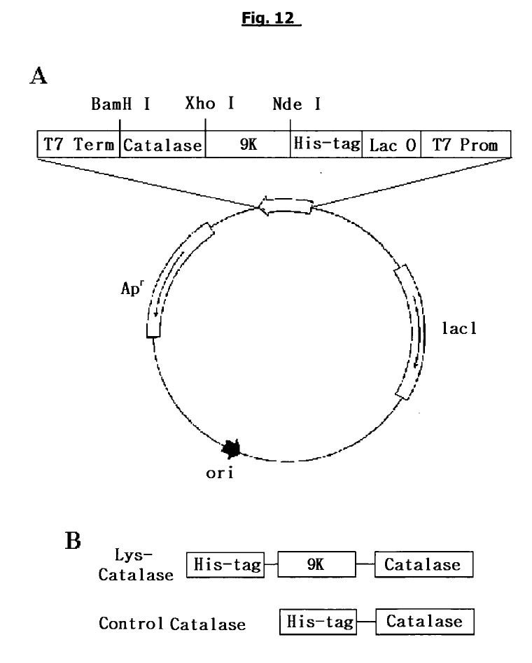

15. The expression vector producing the oligolysine-cargo molecule complex as claimed in claim 13 or 14, wherein the expression vector is a vector shown in FIG. 1B or 12.

16. A method for introducing an oligolysine-cargo molecule complex into a cell or a cell nucleus, comprising the steps of:
(a) expressing the expression vector according to any one of claims 13 to 15 in a microorganism;
(b) purifying the oligolysine-cargo molecule complex expressed; and
(c) adding the purified oligolysine-cargo molecule complex to the cell or the cell nucleus.

17. The method as claimed in claim 16, wherein the step (b) comprises purifying the oligolysine-cargo molecule complex in a denatured form.

18. The method as claimed in claim 17, wherein the oligolysine-cargo molecule complex is denatured by using 4 to 6 M of urea.

19. A cosmetic composition comprising the oligolysine-cargo molecule complex according to claim 5 or the oligolysine fursion protein according to claims 6 to 8 as an effective component.

20. The cosmetic composition as claimed in claim 19, the oligolysinecargo molecule complex is a Lys-SOD fused protein or Lys-CAT and bas an activity of removing active oxygen species.

21. The cosmetic composition as claimed in claim 19 or 20, wherein the cosmetic composition is cosmetic water, gel, aqueous liquid, or oil-in-water (O/W) or water-in-oil (W/O) type cosmetic.


SEQUENCE LISTING (13 sequences)

SEQ ID NO: 1 - DNA, 29 bp, Artificial Sequence
  coding sequence of 9 lysine residues
taagaagaaaaagaaaaagaaaaagaagc

SEQ ID NO: 2 - protein, 9 aa, Artificial Sequence
  9 lysine transducing domain
KKKKKKKKK

SEQ ID NO: 3 - DNA, 757 bp, Artificial Sequence
  polynucleotide coding 9Lys-GFP fusion protein
taagaagaaaaagaaaaagaaaaagaagctcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcc

SEQ ID NO: 4 - protein, 249 aa, Artificial Sequence
  9 lysine-GFP fusion protein
KKKKKKKKKLEVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - DNA, 31 bp, Artificial Sequence
  reverse primer of polynucleotide coding 9 lysine residues
tcgagcttctttttctttttctttttcttct

SEQ ID NO: 6 - DNA, 27 bp, Aequorea victoria
ctcgaggtgagcaagggcgaggagctg

SEQ ID NO: 7 - DNA, 33 bp, Aequorea victoria
ggatccttacttgtacagctcgtccatgccgag

SEQ ID NO: 8 - DNA, 31 bp, Homo sapiens
ggatcctcacagatttgccrrctcccttgcc

SEQ ID NO: 9 - DNA, 30 bp, Homo sapiens
ctcgagatggctgacagccgggatcccgcc

SEQ ID NO: 10 - DNA, 1624 bp, Artificial Sequence
  polynucleotide coding 9lysine-CAT fusion protein
taagaagaaaaagaaaaagaaaaagaagctcgagatggctgacagccgggatcccgccagcgaccagatgcagcactggaaggagcagcgggccgcgcagaaagctgatgtcctgaccactggagctggtaacccagtaggagacaaacttaatgttattacagtagggccccgtgggccccttcttgttcagaatgtggttttcactgatgaaatggctcattttgaccgagagagaattcctgagagagttgtgcatgctaaaggagcaggggcctttggctactttgaggtcacacatgacattaccaaatactccaaggcaaaggtatttgagcatattggaaagaagactcccatcgcagttcggttctccactgttgctggagaatcgggttcagctgacacagttcgggaccctcgtgggtttgcagtgaaattttacacagaagatggtaactgggatctcgttggaaataacacccccattttcttcatcagggatcccatattgtttccatcttttatccacagccaaaagagaaatcctcagacacatctgaaggatccggacatggtctgggacttctggagcctacgtcctgagtctctgcatcaggtttctttcttgttcagtgatcgggggattccagatggacatcgccacatgaatggatatggatcacatactttcaagctggttaatgcaaatggggaggcagtttattgcaaattccattataagactggccagggcatcaaaaacctttctgttgaagatgcggcgagactttcccaggaagatcctgactatggcatccgggatctttttaacgccattgccacaggaaaggacccctcctggactttttacatccaggtcatgacatttaatcaggcagaaacttttccatttaatccattcgatctcaccagggtttggcctcacaaggactaccctctcatcccagttggtaaactggtcttaaaccggaatccagttaattactttgctgaggttgaacagatagccttcgacccaagcaacatgccacctggcattgaggccagtcctgacaaaatgcttcagggccgcctttttgcctatcctgacactcaccgccatcgcctgggacccaattatcttcatatacctgtgaactgtccctaccgtgctcgagtggccaactaccagcgtgacggcccgatgtgcatgcaggacaatcagggtggtgctccaaattactaccccaacagctttggtgctccggaacaacagccttctgccctggagcacagcatccaatattctggagaagtgcggagattcaacactgccaatgatgataacgttactcaggtgcgggcattctatgtgaacgtgctgaatgaggaacagaggaaacgtctgtgtgagaacattgccggccacctgaaggatgcacaaattttcatccagaagaaagcggtcaagaacttcactgaggtccaccctgactacgggagccacatccaggctcttctggacaagtacaatgctgagaagcctaagaatgcgattcacacctttgtgcggtccggatctcacttggtggcaagggagaaggcaaatctgtgaggatcc

SEQ ID NO: 11 - protein, 538 aa, Artificial Sequence
  9 lysine-CAT fusion protein
KKKKKKKKKLEMADSRDPASDQMQHWKEQRAAQKADVLTTGAGNPVGDKLNVITVGPRGPLLVQNVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTGQGIKNLSVEDAARLSQEDPDYGIRDLFNAIATGKDPSWTFYIQVMTFNQAETFPFNPFDLTRVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVRSGSHLVAREKANL

SEQ ID NO: 12 - DNA, 27 bp, Homo sapiens
ctcgaggcgacgaaggccgtgtgcgtg

SEQ ID NO: 13 - DNA, 27 bp, Homo sapiens
ggatccttattgggcgatcccaattac